(19) United States
(12) Patent Application Publication
(10) Pub. No.: US 2009/0281351 A1
(43) Pub. Date: Nov. 12, 2009

(54) NOVEL ALKOXYAMINES, PROCESS FOR THE PREPARATION THEREOF AND USE THEREOF IN RADICAL

(76) Inventors: Jean-Luc Couturier, Lyon (FR); Manuel Hidalgo, Brignais (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/281,378

(22) PCT Filed: Mar. 2, 2007
(86) PCT No.: PCT/EP2007/052018

§ 371 Date: Dec. 19, 2008

(30) Foreign Application Priority Data
Mar. 2, 2006 (FR) 0601879

(51) Int. Cl. C07C 205/04

(52) U.S. Cl. 562/433; 422/129

(57) ABSTRACT

The invention relates to an alkoxyamine of formula (I) below:

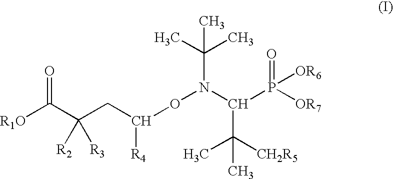

in which R₁ may be a hydrogen atom, R₂ and R₃ alkyl groups, such as a methyl group, R₄ a phenylene group bearing an —SO₃Na group, R₅ a hydrogen atom, and R₆ and R₇ alkyl groups, such as an ethyl group.

Use of these alkoxyamines as a radical polymerization initiator.

### TECHNICAL FIELD

The present invention relates to novel alkoxyamines, that can be used as initiators of radical polymerization, in particular in an aqueous medium.

Patent application FR 2843394 describes alkoxyamines, such as 2-methyl-2-[N-tert-butyl-N-(di-ethoxyphosphoryl-2,2-dimethylpropyl)aminoxy]propionic acid, corresponding to the following general formula:

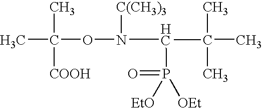

which, when they are used as initiators of a radical polymerization or copolymerization, provide excellent control of the polydispersity while ensuring a good polymerization or copolymerization rate.

However, the use of these alkoxyamines can have a certain number of drawbacks.

Thus, the alkoxyamines described in FR 2843394 must be used in a form which is neutralized with an excess of strong base so as to be water-soluble. In order for it to be possible to use them for the polymerization or copolymerization of water-soluble monomers, it is therefore necessary to carry out, before the polymerization or copolymerization, a step of preparation of the alkoxyamine (for example by neutralization with a strong base).

The applicant has discovered, surprisingly, novel alkoxyamines which make it possible to overcome the drawbacks of those of the prior art, and has discovered, in particular, alkoxyamines which are especially water-soluble irrespective of the pH and which can be used in any type of controlled radical polymerization process, in particular in an aqueous medium, including in the presence of acid monomers such as methacrylic acid and including in acidic media such as those often used in emulsion polymerization.

Thus, according to a first subject, the invention relates to an alkoxyamine of formula (I) below:

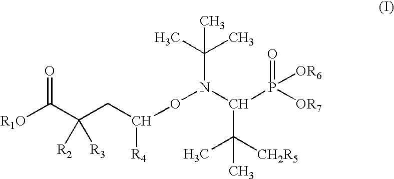

in which:

R₁ represents a hydrogen atom, a linear or branched alkyl group containing from 1 to 8 carbon atoms, a phenyl group, a metal chosen from alkali metals, alkaline earth metals and transition metals, in particular an alkali metal (Na, Li, K), or else H₄N⁺, Bu₄N⁺ or Bu₃HN⁺, Bu representing an n-butyl group;

R₂ and R₃, which may be identical or different, represent a linear or branched alkyl group containing from 1 to 3 carbon atoms;

R₅ represents a hydrogen atom or an —OCOR₈ group, R₈ representing a linear or branched alkyl group containing from 1 to 20 carbon atoms;

R₆ and R₇ represent, independently, a linear or branched alkyl group containing from 1 to 3 carbon atoms;

R₄ represents:

an aryl group bearing at least one acid group comprising at least one heteroatom chosen from S and P, it being possible for said acid group to exist in the form of a salt; or

a heterocyclic group comprising one or more heteroatoms chosen from O, N and/or S, said heterocyclic group optionally bearing at least one acid group comprising at least one heteroatom chosen from S and P or bearing a hydrocarbon-based group optionally comprising one or more heteroatoms (for example N, S and/or O), said hydrocarbon-based group bearing at least one acid group as defined above, it being possible for said heterocyclic group to exist in the form of a salt; or

a —CO—NR—Y or —CO—O—Y group, with Y representing a hydrocarbon-based group optionally comprising one or more heteroatoms (for example, N, S and/or O), bearing at least one acid group comprising a heteroatom chosen from S and P, or representing a hydrocarbon-based group optionally comprising one or more heteroatoms (for example N, S and/or O) containing at least one heterocyclic group comprising one or more heteroatoms chosen from N, O and S, it being possible for said —CO—NR—Y or —CO—O—Y group to optionally exist in the form of a salt, and R representing a hydrogen atom or an alkyl group preferably containing from 1 to 24 carbon atoms.

The alkoxyamines of the present invention may be water-soluble, water-dispersible and/or organosoluble. When reference is made to solubility in a medium, this is intended to mean that the alkoxyamine is soluble in a proportion of at least 1% by weight in this medium.

Thus, according to a first alternative, R₄ may be an aryl group containing, for example, from 5 to 20 carbon atoms (for example, a phenyl group), said aryl group bearing (i.e. being substituted with) at least one acid group comprising at least one heteroatom chosen from S and P, it being possible for said acid group to exist in the form of a salt. By way of example of an acid group comprising at least one heteroatom chosen from S and P, mention may be made of a sulphonic, phosphonic, phosphoric or phosphinic group, and the salts thereof.

According to a second alternative, R₄ may be a heterocyclic group comprising one or more heteroatoms chosen from O, N and/or S, such as a pyrrole, pyridine, indole, thiophene, furan or pyrimidine group, optionally bearing an acid group comprising at least one heteroatom chosen from S or P as defined above, or said heterocyclic group bearing at least one hydrocarbon-based group, such as an alkyl group containing from 1 to 24 carbon atoms, optionally comprising one or more heteroatoms, which hydrocarbon-based group is substituted one or more times with an acid group as defined above (i.e. an acid group comprising at least one heteroatom chosen from S and P).

According to a third alternative, R₄ may represent a —CO—NR—Y or —CO—O—Y group, with Y representing a hydrocarbon-based group, such as an alkyl group which may contain from 1 to 24 carbon atoms, optionally comprising one or more heteroatoms, and substituted with at least one acid group comprising at least one heteroatom chosen from S and P, such as a sulfonic, phosphonic, phosphoric or phosphinic group. By way of example of such a group Y, mention may be made of the group —C(CH₃)₂—CH₂—SO₃H. Y may also be a hydrocarbon-based group, such as an alkyl group containing from 1 to 24 carbon atoms, optionally comprising one or more heteroatoms, and bearing at least one heterocyclic group comprising one or more heteroatoms chosen from N, O, and S, such as an imidazole, imidazoline, imidazolidone, pyrazole, triazole, tetrazole, thiadiazole or oxadiazole group. The group Y cannot be an unsubstituted alkyl group, in so far as it is necessarily substituted with an acid group or a heterocyclic group as defined above.

Among the alkoxyamines of formula (I), use is preferably made most particularly of those for which R₄ is an aryl group bearing at least one acid group comprising at least one heteroatom chosen from S and P, it being possible for said acid group to exist in the form of a salt. In particular, R₄ may advantageously be a phenylene group bearing an —SO₃R₉ group, R₉ representing a hydrogen atom, a metal chosen from alkali metals, alkaline earth metals and transition metals, and in particular an alkali metal (Na, Li, K), or else H₄N⁺, Bu₄N⁺ or Bu₃HN⁺, Bu representing an n-butyl group.

A particular alkoxyamine in accordance with the invention corresponds to formula (II) below:

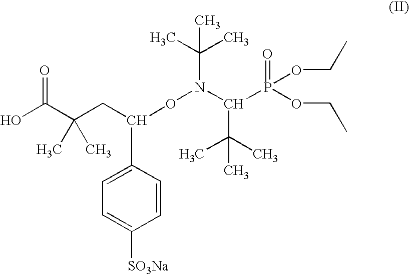

The alkoxyamines of the invention can be obtained by radical addition of the 1,2-type on an olefin comprising a group R₄ as defined above.

Thus, according to a second subject, the invention relates to a process for preparing an alkoxyamine of formula (I) below:

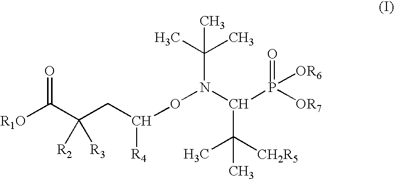

R₁ to R₇ being as defined above, said process comprising a step of reacting an alkoxyamine of formula (III) below:

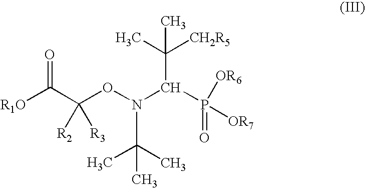

with a compound of formula (IV) below:

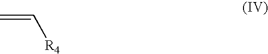

R₄ corresponding to the same definition as that given above.

Without wishing to be bound to the theory, the step of reacting compound (III) with compound (IV) is carried out according to the following reaction scheme:

a) cleavage of compound (III) into free radicals:

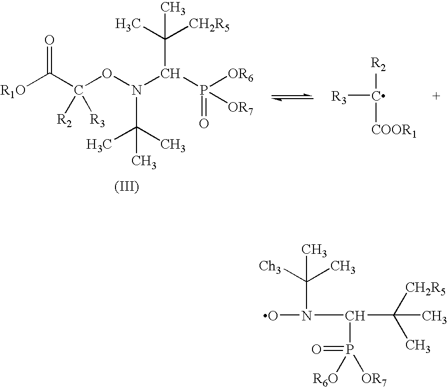

b) 1,2-type radical addition on compound (IV):

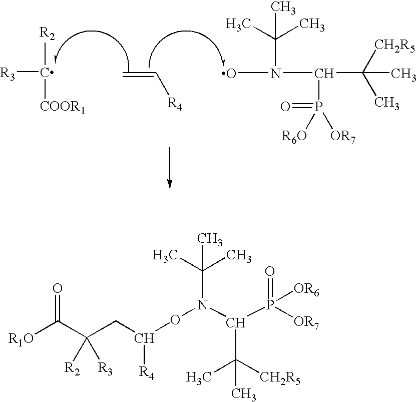

This reaction step is carried out generally in the presence of a solvent, it being possible for this solvent to be chosen from water, methanol, ethanol, isopropanol, acetonitrile, tetrahydrofuran and 1,4-dioxane, or a mixture thereof, at a temperature that may be between 0 and 80° C.

The molar ratio between the alkoxyamine of formula (III) and the compound of formula (IV) can range from 0.9 to 1.5, preferably from 1 to 1.1.

The alkoxyamine of formula (I) obtained can be used as it is in solution or can, over the course of a final step, be isolated by evaporation of the solvent or by precipitation by adding a nonsolvent.

For the purpose of preparing the alkoxyamine of formula (I), a reservoir comprising two compartments, comprising respectively the alkoxyamine (III) and the compound of formula (IV), may be envisaged, it being possible for the content of each of these compartments to be mixed when desired for the preparation of the alkoxyamine of formula (I).

Thus, according to a third subject, the invention relates to a reservoir for the purpose of preparing an alkoxyamine of formula (I) as defined above, comprising:
a first compartment comprising an alkoxyamine of formula (III) as defined above; and
a second compartment comprising a compound of formula (IV) as defined above.

Finally, according to a fourth subject, the invention relates to the use of an alkoxyamine of formula (I) as defined above, as a radical polymerization initiator.

The invention will now be described with reference to the following example, given by way of nonlimiting illustration.

### DETAILED DISCLOSURE OF A PARTICULAR EMBODIMENT

### Example

This example illustrates the preparation of an alkoxyamine of formula (II) below:

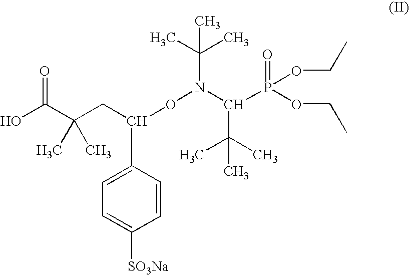

from an alkoxyamine of the formula below:

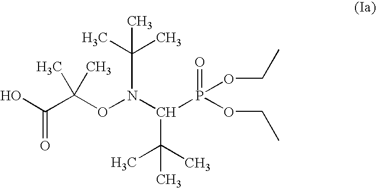

and sodium 4-styrenesulfonate:

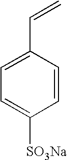

1 litre of ethanol and 0.5 litre of degassed water are introduced in a 2-litre glass reactor flushed with nitrogen. 54 g of sodium 4-styrenesulfonate (0.262 mol) and 100 g of alkoxyamine of formula (Ia) (0.262 mol) are added. The mixture is heated to 70° C. and left to react for 6 hours with stirring. The product is recovered by evaporation under vacuum at a temperature of 30° C. 179 g of a yellow oil are obtained, which oil crystallizes upon storage at 4° C. in the form of a wax.

The product obtained is analysed by ¹H, ¹³C and ³¹P NMR, by negative electrospray mass spectrometry and by Karl Fischer analysis. It contains 84% by weight of alkoxyamine of formula (II) in the form of 2 diastereoisomers in 59/41 proportions and 16% of water.

The characteristics of the alkoxyamine obtained are the following:

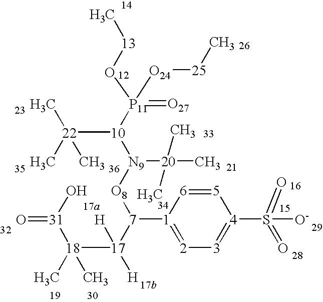

### —¹H NMR (CDCl₃)

| Atom No. | Chemical shift (in ppm) |
|---|---|
| 2 | 7.45 |
| 3 | 7.7 |
| 5 | 7.7 |
| 6 | 7.45 |
| 7 | 5.1 |
| 19 | 1.13 |
| 30 | 1.13 |
| 10 | 3.4 |
| 17a | 2.3 |
| 17b | 2.5 |
| 21 | 0.8 |
| 23 | 1.10-1.21 |
| 33 | 0.8 |
| 34 | 0.8 |
| 35 | 1.10-1.21 |
| 36 | 1.10-1.21 |
| 25 | 3.2 |
| 13 | 3.65-4.45 |
| 26 | 0.9 |
| 14 | 1.20-1.40 |

Major isomer

| Atom No. | Chemical shift (in ppm) |
|---|---|
| 7 | 4.9 |
| 6 | 7.3 |
| 2 | 7.3 |
| 5 | 7.7 |
| 3 | 7.7 |
| 10 | 3.32 |
| 23 | 1.10-1.22 |
| 35 | 1.10-1.22 |
| 36 | 1.10-1.22 |
| 26 | 0.9 |
| 13 | 3.65-4.45 |
| 14 | 1.20-1.40 |
| 17 | 2.50-2.75 |
| 19 | 1.1 |
| 21 | 1.19 |
| 25 | 3.65-4.45 |
| 30 | 1.1 |
| 33 | 1.19 |
| 34 | 1.19 |

- ¹³C NMR (CDCl₃)

Minor isomer

| Atom No. | Chemical shift (in ppm) |
|---|---|
| 1 | 141.60-143.40 |
| 2 | 129.50 |
| 3 | 125.00 |
| 4 | 141.60-143.40 |
| 5 | 125.00 |
| 6 | 129.50 |
| 7 | 79.80 |
| 10 | 69.50 |
| 13 | 61.50 |
| 14 | 15.95-16.50 |
| 17 | 43.00 |
| 18 | 39.72 |
| 19 | 22.30 |
| 20 | 61.20 |
| 21 | 28.20 |
| 22 | 35.00 |
| 23 | 30.20 |
| 25 | 58.70 |
| 26 | 15.95-16.50 |
| 30 | 22.30 |
| 31 | 179.80 |
| 33 | 28.20 |
| 34 | 28.20 |
| 35 | 30.20 |
| 36 | 30.20 |

Major isomer

| Atom No. | Chemical shift (in ppm) |
|---|---|
| 1 | 141.60-143.40 |
| 2 | 128.80 |
| 3 | 124.60 |
| 4 | 141.60-143.40 |
| 5 | 124.60 |
| 6 | 128.80 |
| 7 | 86.60 |
| 10 | 69.40 |
| 13 | 61.50 |
| 14 | 15.95-16.50 |
| 17 | 45.20 |
| 18 | 39.69 |
| 19 | 21.80 |
| 20 | 60.90 |
| 21 | 28.20 |
| 22 | 35.30 |
| 23 | 30.20 |
| 25 | 58.70 |
| 26 | 15.95-16.50 |
| 30 | 21.80 |
| 31 | 179.80 |
| 33 | 28.20 |
| 34 | 28.20 |
| 35 | 30.20 |
| 36 | 30.20 |

- ³¹P NMR (CDCl₃)
Major isomer: 24 ppm
Minor isomer: 25 ppm

Minor isomer

1. Alkoxyamine of formula (I) below: 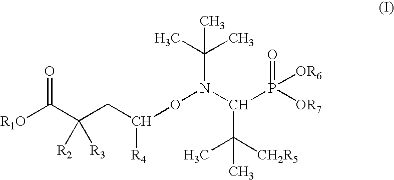
in which:
R₁ represents a hydrogen atom, a linear or branched alkyl group containing from 1 to 8 carbon atoms, a phenyl group, a metal chosen from alkali metals, alkaline earth metals and transition metals, H₄N⁺, Bu₄N⁺ or Bu₃HN⁺, Bu representing an n-butyl group;
R₂ and R₃, which may be identical or different, represent a linear or branched alkyl group containing from 1 to 3 carbon atoms;
R₅ represents a hydrogen atom or an —OCOR₈ group, R₈ representing a linear or branched alkyl group containing from 1 to 20 carbon atoms;
R₆ and R₇ represent, independently, a linear or branched alkyl group containing from 1 to 3 carbon atoms;
R₄ represents:
an aryl group bearing at least one acid group comprising at least one heteroatom chosen from S and P, it being possible for said acid group to exist in the form of a salt; or
a heterocyclic group comprising one or more heteroatoms chosen from O, N and/or S, said heterocyclic group optionally bearing at least one acid group comprising at least one heteroatom chosen from S and P or bearing a hydrocarbon-based group optionally comprising one or more heteroatoms, said hydrocarbon-based group bearing at least one acid group as defined above, it being possible for said heterocyclic group to exist in the form of a salt; or
a —CO—NR—Y or —CO—O—Y group, with Y representing a hydrocarbon-based group optionally comprising one or more heteroatoms, bearing at least one acid group comprising a heteroatom chosen from S and P, or representing a hydrocarbon-based group optionally comprising one or more heteroatoms containing at least one heterocyclic group comprising one or more heteroatoms chosen from N, O, and S, it being possible for said —CO—NR—Y or —CO—O—Y group to optionally exist in the form of a salt, and R representing a hydrogen atom or an alkyl group.

2. Alkoxyamine according to claim 1, in which R₄ is an aryl group bearing at least one sulfonic, phosphonic, phosphoric or phosphinic group, it being possible for these groups to exist in the form of salts.

3. Alkoxyamine according to claim 1, in which R₄ is a pyrrole, pyridine, indole, thiophene, furan or pyrimidine group.

4. Alkoxyamine according to claim 1, in which R₄ represents a —CO—NR—Y or —CO—O—Y group, with Y representing a hydrocarbon-based group bearing at least one imidazole, imidazoline, imidazolidone, pyrazole, triazole, tetrazole, thiadiazole or oxadiazole group.

5. Alkoxyamine according to claim 1, in which R₄ is a phenylene group bearing an —SO₃R₉ group, R₉ representing a hydrogen atom, a metal chosen from alkali metals, alkaline earth metals and transition metals, H₄N⁺, Bu₄N⁺ or Bu₃HN⁺, Bu representing an n-butyl group.

6. Alkoxyamine according to claim 5, corresponding to formula (II) below:

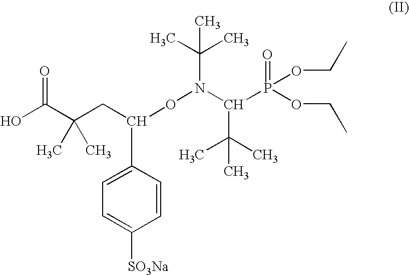

7. Process for preparing an alkoxyamine of formula (I) below: 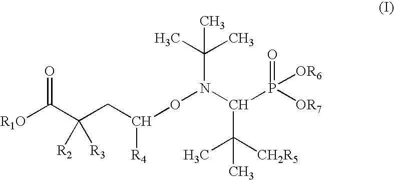
in which:
R₁ represents a hydrogen atom, a linear or branched alkyl group containing from 1 to 8 carbon atoms, a phenyl group, a metal chosen from alkali metals, alkaline earth metals and transition metals, H₄N⁺, Bu₄N⁺ or Bu₃HN⁺, Bu representing an n-butyl group;
R₂ and R₃, which may be identical or different, represent a linear or branched alkyl group containing from 1 to 3 carbon atoms;
R₅ represents a hydrogen atom or an —OCOR₈ group, R₈ representing a linear or branched alkyl group containing from 1 to 20 carbon atoms;
R₆ and R₇ represent, independently, a linear or branched alkyl group containing from 1 to 3 carbon atoms;
R₄ represents:
an aryl group bearing at least one acid group comprising at least one heteroatom chosen from S and P, it being possible for said group to exist in the form of a salt; or
a heterocyclic group comprising one or more heteroatoms chosen from O, N and/or S, said heterocyclic group optionally bearing at least one acid group comprising at least one heteroatom chosen from S and P or bearing a hydrocarbon-based group optionally comprising one or more heteroatoms, said hydrocarbon-based group bearing at least one acid group as defined above, it being possible for said heterocyclic group to exist in the form of a salt; or
a —CO—NR—Y or —CO—O—Y group, with Y representing a hydrocarbon-based group optionally comprising one or more heteroatoms bearing at least one acid group comprising a heteroatom chosen from S and P, or representing a hydrocarbon-based group optionally comprising one or more heteroatoms bearing at least one heterocyclic group comprising one or more heteroatoms chosen from N, O and S, it being possible for said —CO—NR—Y or —CO—O—Y group to optionally exist in the form of a salt, and R representing a hydrogen atom or an alkyl group, said process comprising a step of reacting an alkoxyamine of formula (III) below: 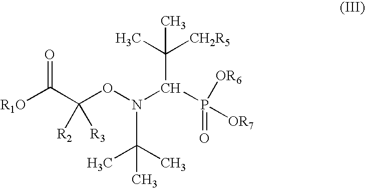
with a compound of formula (IV) below: 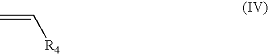
R₁ to R₇ being as defined above.

8. Process according to claim 7, in which the reaction step is carried out in the presence of a solvent chosen from water, methanol, ethanol, isopropanol, acetonitrile, tetrahydrofuran and 1,4-dioxane, or a mixture thereof.

9. Process according to claim 7, in which the molar ratio between the alkoxyamine of formula (III) and the compound of formula (IV) ranges from 1 to 1.1.

10. Reservoir for the purpose of preparing an alkoxyamine of formula (I) according to claim 7, comprising:
a first compartment comprising an alkoxyamine of formula (III) as defined in claim 7; and
a second compartment comprising a compound of formula (IV) as defined in claim 7.

11. Use of an alkoxyamine of formula (I) as defined in claim 1, as a radical polymerization initiator.

12. A process for performing a radical polymerization, said process comprising performing radical polymerization in the presence of an alkoxyamine according to claim 1 as a radical initiator.
